(19) United States
(12) Patent Application Publication
(10) Pub. No.: US 2014/0024949 A1
(43) Pub. Date: Jan. 23, 2014

(54) SURGICAL MICROSCOPY SYSTEM INCLUDING AN OCT SYSTEM

(71) Applicant: Carl Zeiss Meditec AG, Jena (DE)

(72) Inventors: Xing WEI, Dublin, CA (US); Christoph HAUGER, Aalen (DE); Holger MATZ, Unterschneidheim (DE)

(21) Appl. No.: 14/035,323

(22) Filed: Sep. 24, 2013

Related U.S. Application Data
(63) Continuation of application No. PCT/EP2012/001373, filed on Mar. 26, 2012.

(30) Foreign Application Priority Data
Mar. 25, 2011 (DE) 10 2011 015 149.4

(51) Int. Cl. A61B 5/00

(52) U.S. Cl. CPC A61B 5/7425; A61B 5/0066; A61B 5/0035
USPC 600/476

(57) ABSTRACT

Receiver adapted for determining an estimation of interferences when receiving an OFDM signal made of packets, each packet comprising a first training field, a second training field, at least two header fields and data field, comprising:
Means for detecting a first symbol value of a first header fields and a second symbol value of a second header field, said first and second header fields beholding to said at least two header fields and the modulation scheme being different between said first and second header fields; and
Means for determining said estimation from said first and second symbol values.

The present invention relates to a surgical microscope system having imaging optics for imaging an object region, wherein the surgical microscope system is combined with an OCT-system for obtaining measurement data by optical coherence tomography.

### BACKGROUND

From US 2009/0257065 A1, there is known a surgical microscopy system having an integrated OCT-system. Such a surgical microscopy system allows to optically image an object field, wherein the optically generated image is observable through eyepieces of the imaging optical system. An OCT-system, which is integrated into the surgical microscopy system, is configured to scan an OCT measuring beam across the object field and to conducting measurements by means of optical coherence tomography. To this end, the system comprises a beam deflector for deflecting the OCT-measurement beam and directing the OCT-measuring beam to locations in the object field for conducting OCT-measurements.

For users of such systems, it is difficult to select locations at which OCT measurements are to be conducted and to correlate the obtained OCT data with images obtained by the imaging optics.

Accordingly, there is a need for a surgical microscope system having imaging optics and an OCT-system, and which allows easy selection of locations at which OCT-measurements are to be taken.

Also, there is a need for a surgical microscope system having imaging optics and an OCT-System, and which allows to interpret results of the OCT-measurements more accurately.

These objects are achieved by the independent claim. Further embodiments are specified in the dependent claims.

### SUMMARY

Embodiments provide a surgical microscope system comprising: imaging optics for imaging a portion of an object field of the imaging optics onto a camera sensor, wherein the imaging optics comprises a zoom lens arrangement for varying a magnification of the imaging optics for the imaging of the portion of the object field onto the camera sensor; an OCT-system for generating an OCT measurement beam for performing measurements by optical coherence tomography; a beam deflector for deflecting the OCT measurement beam and guiding the OCT measurement beam to selectable locations in the object field; a graphical user interface for displaying in a drawing area of the graphical user interface an image of the portion of the object field detected by the camera sensor; a first control module for controlling the beam deflector and the OCT system such that the OCT measurement beam is guided along an adjustable scanning path over the object field and such that OCT measurements are taken at a plurality of locations on the scanning path; a second control module for determining the scanning path, wherein the second control module is configured to display the scanning path in the drawing area of the graphical user interface, wherein coordinates of points, which represent the scanning path in the drawing area are determined in dependence on deflecting angles of the beam deflector, which correspond to the scanning path, and further in dependence on the magnification of the imaging optics.

By displaying the selectable scanning path in the same drawing area of the graphical user interface, in which the image, detected by the camera sensor, is displayed, it is easy for the user to understand, which portions of the object are scanned with the OCT-measurement beam. Also thereby, it is possible for the user to vary a parameter of the scanning path, in case he considers this necessary.

The imaging optics may be configured to generate an image of the portion of the object field in an image plane of the imaging optics. The image plane may be located at a detecting surface of the camera sensor. In other words, the imaging optics may be configured to generate the image of the portion of the object field without applying a scanning technique.

The scanning path may comprise one or more straight or curved lines. In case the scanning path comprises a plurality of lines, at least a portion of the plurality of lines may be connected end to end. The scanning path may be located within a plane, which is oriented perpendicular to an axis of the OCT measurement beam. The axis of the OCT measurement beam may be oriented parallel or substantially parallel to an optical axis of the imaging optics. It is also conceivable that the axis of the OCT measurement beam and the optical axis of the imaging optics form an angle, which is greater than zero.

The object field may be located in a plane perpendicular to the optical axis of the imaging optics. The optical axis of the imaging optics may be an optical axis of an objective lens of the imaging optics. The scanning path may be located within the object field or within the portion of the object field, which is imaged onto the camera sensor.

The OCT system and the beam deflector may be configured to perform OCT depth scans at one or more locations on the scanning path. The depth scans may be acquired along a direction which is parallel the axis of the OCT measurement beam.

The coordinates may be determined depending on the current magnification of the imaging optics.

According to an embodiment, the graphical user interface comprises at least one control element for moving the scanning path relative to the object field.

A control element may be defined as a component of the graphical user interface, which is configured to receive input from the user. The control element may be configured to receive user input comprising a direction and/or a length of a translatory movement of the scanning path relative to the object field. A displacement vector of the movement may be located within the object field of the imaging optics. The movement may be in a direction perpendicular to an optical axis of the imaging optics or an optical axis of an objective lens of the imaging optics.

According to a further embodiment, the graphical user interface comprises at least one control element for performing a translatory and/or rotatory movement of the scanning path relative to the object field. The translatory and/or rotatory movement may be within the object field.

According to a further embodiment, the graphical user interface comprises at least one control element for varying a lateral extend of the scanning path relative to the object field.

The graphical user interface may be configured such that at least one scaling factor is settable by user input via the graphical user interface. The scaling factor may be a one-dimensional scaling factor or a two-dimensional scaling factor. By way of example, the graphical user interface may be configured such that the scanning path is scalable along an axis, which is located parallel to the object field. The direction of the axis may be adjustable by a user via the graphical user interface. Additionally or alternatively, the surgical microscope system may be configured such that a two-dimensional scaling factor is settable by the user via the graphical user interface. The two-dimensional scaling factor may scale the scanning path in two dimensions. The one-dimensional scaling and the two-dimensional scaling may be relative to the object field. Lateral extent may be defined as an extent measured within the object field.

According to a further embodiment, the second control module is configured to adjust a geometry of the scanning path depending on a user input received via the graphical user interface.

Adjusting the scanning path may comprise at least one of the following: adjusting a one- or two-dimensional scaling factor of the scanning path relative to the object field, translationally moving the scanning path in a direction parallel to the object field, rotating the scanning path around an axis perpendicular to the object field, translationally moving an end point of a line of the scanning path in a direction parallel to the object field, translationally moving a line of the scanning path in a direction parallel to the object field and rotating a line of the scanning path around an axis perpendicular to the object field. The end point of a line may be a connecting point between two lines of the scanning path.

According to a further embodiment, the graphical user interface is configured to display the points, which represent the scanning path, superimposed on the displayed image of the portion of the object field.

Accordingly, it is possible for the user to correlate features in the image of the portion of the object field acquired by the imaging optics with OCT data of the OCT measurements. For example, it is possible for a user to see, whether an OCT scanning path traverses a defect on an object surface, which is visible in the image of the portion of the object. Thereby, the user can adjust the scanning path such that a region of interest is measured by OCT.

The second control module may be configured to determine the coordinates of the points, which represent the scanning path, such that for each of the points, a location of the point is at a pixel of the image detected by the camera sensor, which corresponds to the same location in the portion of the object field, as the point. In other words, the points representing the scanning path are located at pixels, which correspond to locations in the object field, where the scanning path is located.

According to a further embodiment, the graphical user interface is configured to display in the drawing area the plurality of locations on the scanning path, where the OCT measurements are taken.

The OCT system may be configured to conduct at the plurality of locations on the scanning path OCT measurements. The locations of the OCT measurements may be displayed superimposed on the image of the portion of the object field. The locations of the OCT measurements may be displayed by a representation, such as an icon or a point. The displayed locations in the drawing area may be located at pixels of the image, acquired with the camera sensor, wherein the locations of the pixels correspond to the locations, where the OCT measurements have been acquired. The locations of the OCT measurements may be displayed superimposed on the points representing the scanning path. The OCT measurements may be OCT depth scans. An OCT depth scan may be referred to as an A-scan. The plurality of OCT depth scans on the scanning path may represent a B-Scan.

According to an embodiment, the surgical microscope is configured such that a number and/or locations of the OCT measurements are settable via the graphical user interface.

According to a further embodiment, the graphical user interface comprises at least one control element for selecting a scanning path type from a plurality of predefined scanning path types.

The graphical user interface may be configured such that the a selected scanning path type is adaptable depending on user input via the graphical user interface.

According to a further embodiment, a scanning path of at least one predefined scanning path types comprises a plurality of scanning lines, which extend linearly and which are laterally spaced from each other.

The scanning lines may be arranged parallel to each other. A distance between neighboring scanning lines may be constant. Laterally spaced may be defined as being arranged spaced apart and arranged in the object field.

According to a further embodiment, the graphical user interface comprises at least one control element for varying a length of the scanning lines.

According to a further embodiment, the graphical user interface comprises at least one control element for varying a lateral distance of the scanning lines.

The lateral distance of the scanning lines may be a lateral distance between neighboring scanning lines. The scanning lines may be oriented parallel with respect to each other. Lateral distance may be defined as being a distance measured along a direction within the object field.

According to a further embodiment, the graphical user interface comprises at least one drawing area for displaying a result of the OCT measurements.

The graphical user interface may be configured to display in the drawing area a plurality of OCT measurements side by side. The plurality of OCT measurements or OCT measurements may represent a B-scan.

According to a further embodiment, the scanning path comprises a plurality of scanning lines, and wherein the graphical user interface comprises a plurality of drawing areas each of which configured to display a result of the OCT-measurements related to one of the plurality of scanning lines.

According to a further embodiment, the second control module is configured to determine the coordinates of the points, which represent the scanning path in the drawing area further in dependence on predefined parameters, which influence at least one of a translation of the coordinates in the drawing area, a rotation of the coordinates in the drawing area, and a scaling of the coordinates in the drawing area.

According to a further embodiment, the graphical user interface comprises at least one control element for adjusting the predefined parameters.

According to a further embodiment, the surgical microscope comprises a calibration object, which comprises a first and a second structure, which are arranged at a predefined position and/or a predefined orientation relative to each other, wherein the first structure is detectable by the camera sensor and the second structure is detectable by the OCT-system.

According to a further embodiment, the surgical microscope comprises a calibration object, comprising at least one structure, wherein the structure is detectable by the camera sensor and by the OCT-system.

According to a further embodiment, the surgical microscope comprises a third control module, which is configured to determine a position and/or an orientation of the first structure in the image detected by the camera sensor and a position and/or an orientation of the second structure in the object field from the OCT measurements and to adjust the predefined parameters in dependence on the determined position and/or orientation of the first structure, the determined position and/or orientation of the second structure and the magnification of the imaging optics.

### BRIEF DESCRIPTION OF THE DRAWINGS

The forgoing as well as other advantageous features will be more apparent from the following detailed description of exemplary embodiments with reference to the accompanying drawings. It noted that not all possible embodiments necessarily exhibit each and every, or any, of the advantages identified herein.

FIG. 1 is a schematic illustration of a surgical microscope system according to an exemplary embodiment; and

FIG. 2 is a schematic illustration of a graphical user interface of the exemplary embodiment shown in FIG. 1.

### DETAILED DESCRIPTION OF EXEMPLARY EMBODIMENTS

In the exemplary embodiments described below, components that are alike in function and structure are designated as far as possible by alike reference numerals. Therefore, to understand the features of the individual components of a specific embodiment, the descriptions of other embodiments and of the summary of the invention should be referred to.

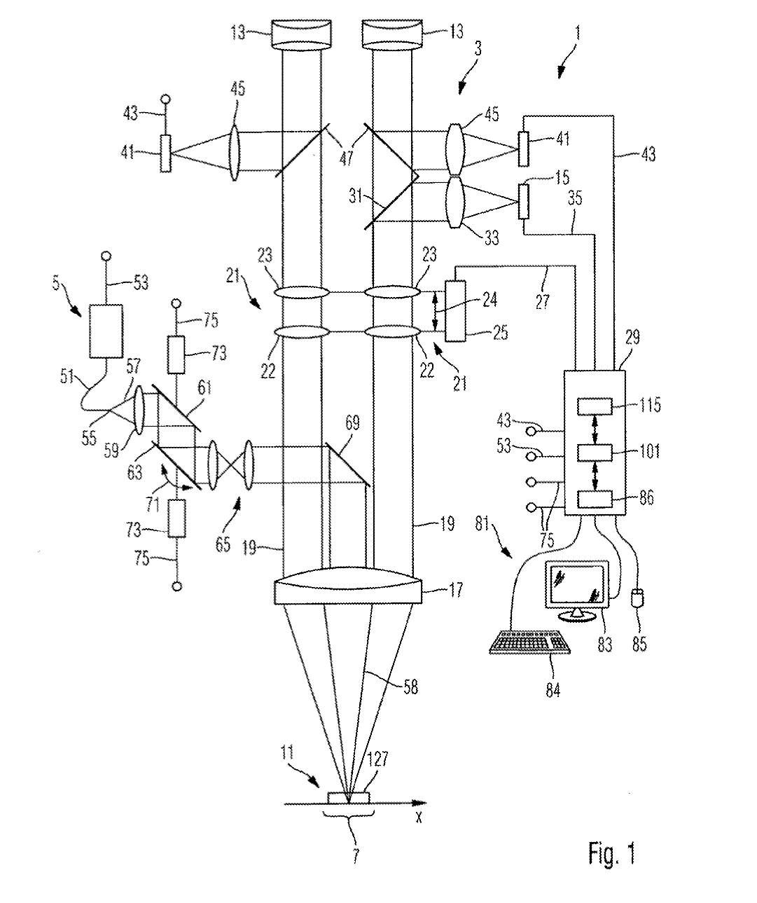

The microscope system, which is schematically illustrated in FIG. 1 comprises imaging optics 3 and an OCT-system 5. The imaging optics 3 are configured to generate optical images of a portion 7 of an object region 11.

In the imaging optics 3 of the illustrated exemplary embodiment, the imaging of the portion 7 of the object region 11 is performed by a pair of eyepieces 13, through which the user looks with his eyes, and further by a camera sensor 15, which is configured to electronically detect an image of the portion 7 of the object region 11. The camera sensor may be an image sensor. By way of example, the camera sensor is a CCD image sensor. A sensor plane of the camera sensor is located in an image plane, which is optically conjugate to the object field 11. To this end, the imaging optics comprise an objective lens 17, which may consist of one or more lens elements and which, in the illustrated example, images the object field 11 to infinity. In the beam path downstream of the objective lens 17, each of two partial beam bundles 19 is guided to a respective zoom lens arrangement 21. The zoom lens arrangements are configured such that a magnification of the imaging optics is variable. To this end, each of the zoom lens arrangements comprises at least two optical components 22, 23, comprising a lens or a lens group, wherein the two optical components are displaceable relative to each other along a beam direction of the partial beam bundle 19, which traverses the two optical components. In FIG. 1, this is illustrated by arrow 24. The displacement of the optical components 22, 23 relative to each other is controlled by an actuator 25, which is again controlled by a controller 29 via a control line 27 for adjusting the magnification of the imaging optics.

After having passed the zoom lens arrangements 21, the partial beam bundles 19 enter the eyepieces 13. From the partial beam bundle 19, which is shown on the right hand side of FIG. 1, a portion of the light of the partial beam bundle is deflected by a partially transmitting mirror 31 and guided via camera adapter optics 33 to a camera sensor 15, such that the camera sensor 15 can detect an image of the portion 7 of the object region 11. Image data, which are generated by the image sensor 15, are transmitted via a data line 35 to the controller 29.

The imaging optics 3 further comprise two electronic displays 41. The controller 29 transmits image data via data lines 43 to the electronic displays 41. Each of the images, which are displayed by the displays 41 is projected by one of a pair of projection optics 45 and by one of a pair of partially transmitting mirrors 47 into one of the beam paths leading to one of the pair of eyepieces 13. Each of the partially transmitting mirrors is arranged in one of the partial beam bundles 19. Thereby, a user, who looks into the eyepieces 13, sees the images, displayed by the displays 41, superimposed onto the image of the portion 7 of the object region 11.

The OCT-system 5 comprises a short coherence light source (white light source) and an interferometer (not illustrated in FIG. 1) for performing the OCT measurements. OCT measurement light is emitted from an optical fiber 51 of the OCT system such that the measuring light is incident onto an object to be inspected and a portion of the measuring light, which returns from the object, enters again into the optical fiber 51. Thereby, it is possible for the OCT-system 5 to analyze the portion of the returning measurement light and to output an OCT-spectrum. The OCT-system 5 is controlled by the controller 29 via control and data line 53. The controller 29 receives OCT measurement data from the OCT system 5 also via the control and data line 53

The OCT measuring light 57, which is emitted from an end 55 of the fiber 51 is collimated by collimating optics 59 to form a measurement beam 58. The measurement beam 58 is deflected by two scan mirrors 61 and 63, traverses projection optics 65, and is incident onto mirror 69. The measurement beam 58 is directed by mirror 69 through objective lens 17 onto the object field 11. A portion of the measuring light, which is reflected by an object, arranged in the object region 11, propagates along an inverse path through the objective lens 17, the projection optics 65 and the collimation optics 59 and is, at least partially, coupled into the optical fiber 51, such that the OCT-system 5 can analyze the returning measurement light.

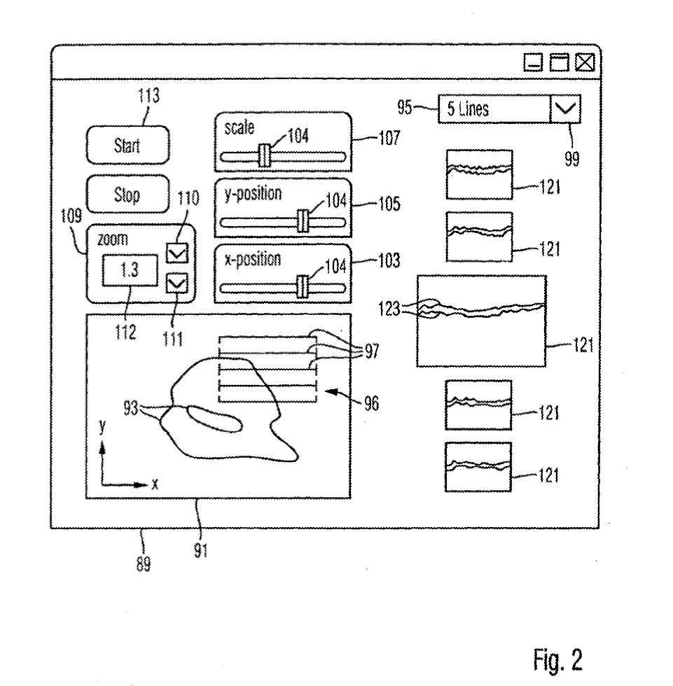

Scan mirror 61 and/or scan mirror 63 may be configured as pivotably mounted mirrors. Scan mirrors 61 and 63 are configured to deflect the OCT measurement beam 58, such that it is incident onto the object field 11 at different locations, depending on the pivoting positions of the scan mirrors 61, 63. As is illustrated by arrow 71 in FIG. 2, scan mirror 63 is pivotable such that a pivoting of the scan mirror causes a displacement of the impingement location of the OCT measuring beam in the object field 11 along the x-direction, i.e. the horizontal direction in the drawing plane of FIG. 1. Accordingly, the scan mirror 61 is pivotable such that a pivoting of the scan mirror 61 causes a displacement of the impingement location of the OCT measuring beam along the y-direction in the object field, i.e. perpendicular to the drawing plane of FIG. 1. The pivoting positions of scan mirrors 61 and 63 are adjusted by actuators 73, which are controlled by the controller 29 via control lines 75. The controller 29 can thereby guide the OCT measurement beam along a selectable scanning path across the object field by controlling the actuators 73.

The surgical microscope system 1 comprises a graphical user interface 81, which comprises a monitor 83 as display medium, a keyboard 84 and a mouse 85 as input devices and a control module 86, which is operated as a software module in the controller 29.

The control module 86 generates an application window 89 on the monitor 83, which is schematically illustrated in FIG. 2. The application window 89 comprises a plurality of control elements and drawing areas. A drawing area may represent an area over which the graphical user interface can draw or otherwise render images, geometric objects and/or text so as to present information to a user. In a first drawing area 91, the control module 86 displays the image of the portion 7 of the object field 11, which has been acquired by the camera sensor 15. Lines 93 in FIG. 2 represent structures of the object, which appear in the image, which has been acquired by the camera sensor 15.

The control element 95 in the application window 89 serves for selecting a scanning path type from a group of predefined scanning path types. In the graphical user interface shown in FIG. 2, a scanning path type is selected. The scanning path 5 of the selected scanning path type comprises scan lines 97, which extend linearly and which are arranged distant from each other. The scan lines 97 may be arranged parallel to each other. In the illustrated example, the control element 95 is configured as a dropdown list, which can be activated by clicking a button with the pointer of the mouse. Thereby, other scan path types, such as for example three parallel scan lines, seven parallel scan lines, concentric circles, or the like, are selectable.

The selected scanning path type is transmitted from control module 86 to control module 101 of the controller 29. Based on the selected scanning path type and depending on further parameters, which are described in the following, the control module 101 generates scanning path data for the scanning path. The scanning path data comprise a sequence of pivoting positions for the scanning mirrors 61 and 63, wherein the pivoting positions are sequentially adopted by the scanning mirrors 61 and 63 for guiding the OCT measurement beam 58 across the object field 11. The scanning path data are transmitted from the control module 101 to the control module 86. Control module 86 displays the scanning path in the drawing area 91 of the application window 89, such that the five scanning lines 97 of the selected scanning path type are visible superimposed over the structures 93 of the object. The xy-coordinates of the points of the drawing area 91, which represent the scanning lines 97, are determined by the control module 101 in dependence on the deflecting angles of the beam deflector 61, 63 for generating the scanning path 96 and further in dependence on the magnification of the imaging optics 3. The magnification of the imaging optics 3 is adjusted by the controller 29 through activating the actuators 25. A variation of the length of the scanning lines 97 in the object field 11 is not varied by a variation of the magnification of the imaging optics. However, a variation of the magnification of the imaging optic 3 results in a variation of the length of the scanning lines 97, as shown in the drawing area 91.

The application window 89 comprises further control elements for adjusting parameters of the selectable scanning path 96. Control element 103 is configured to displace the scanning path in the object field 11 along the x-direction and is implemented as a slide bar, wherein a slide object 104 of the slide bar is graspable and displaceable with the pointer of the mouse 85. A further control element 105 is configured to displace the scanning path in the object field 11 along a y—direction and is also implemented as a slide bar, wherein a slide object 104 is graspable and displaceable by the pointer of the mouse 85. A further control element 107 is configured to vary the size of the scanning path, by scaling all deflecting angles. Also this control element is implemented as a slide bar having a slide object 104.

A control element 109 is configured to vary the magnification of the imaging optics 3. To this end, the user may click a button 110 with the pointer of the mouse 85 for increasing the magnification in a stepwise manner. By clicking a button 111, the user may decrease the magnification in a stepwise manner. Alternatively, the user may input a desired magnification in an input field 112 with the keyboard 84. A further control element 113 is configured to start an OCT measurement. The control element 113 is implemented as a button, which is clickable with the pointer of the mouse 85 for starting the OCT-measurement. After the button has been clicked, a control module 115 of the controller 29 receives data values from the module 101, which represent a scanning path, which has been selected and/or adapted by the user using the control elements 95, 103, 105 and 107. Then, control module 115 controls the actuators 73 of the scanning mirrors 61 and 63, such that the OCT measurement beam is guided across the object field 11 according to the selected and/or adapted scanning path. At each location of a plurality of locations of the scanning path, the OCT system acquires an OCT spectrum and transmits corresponding measurements data to the controller 29. An OCT spectrum may be an OCT depth scan. The OCT spectra of a scanning path or a portion of a scanning path, such as a scanning line may represent an OCT B-Scan. The controller 29 displays the measurement data in drawing areas 121 of the application window 89. To each of the scanning lines 97, a separate drawing area 121 is assigned, such that for the selected scanning path type having five lines, five drawing areas 121 are displayed for displaying the OCT measurements. Each of the five drawing areas 121 is displayable in a magnified manner when selected by the user, far example by clicking the respective drawing area with the pointer of the mouse. In the example shown in FIG. 2, the rendering space 121 in the middle is selected and shown in a magnified fashion by the graphical user interface. In the selected drawing area, shown in FIG. 2, lines 123 are visible, which are caused by layer structures of the object arranged in the object field 11. The OCT-spectra, which are acquired at a plurality of positions along the scanning line 97 are displayed horizontally side by side. In other words, the data shown in each of drawing area 121 represent a vertical cross section of the object. The cross-section is oriented perpendicular to the object field and measured along a portion of the scanning path, wherein the portion is represented by one of the lines 97.

For calibrating the positioning of the scanning path relative to the image of a portion of the object field, which has been acquired by the camera sensor, a calibrating object 127 is provided, which comprises structures, which are detectable by the camera sensor as well as by the OCT system. In case the calibration object is arranged in the center of the object field, the user may recognize the structures 93 of the calibration object in the drawing area 91 as well as in the drawing area 121, showing data of the OCT scan. Then, he can operate the control elements 103, 105 and 107 such that the structures shown in the OCT data coincide with corresponding structures shown in the image, which has been acquired with the camera sensor. These settings of the control elements, which for example correspond to the x-position, the y-position and the scaling factor may be used by the controller 29 such that the control module 101 may determine the xy-coordinates of the points of the scanning path in the drawing area 91 also in dependence of these parameters.

It is also conceivable that the controller 29 comprises a further control module, which analyzes—in a calibration mode of the controller 29—the image, which has been acquired from the calibration object 127 by the camera sensor 15. The further control module determines the orientation of the structures of the calibration object 127 in the image. Then, the OCT measurement beam is guided across the calibration object 127 and the orientation of the structures in the OCT measurement data are determined. The further control module then determines parameters of a coordinate transformation between positions and orientations of the structures in the image, which has been acquired by the camera sensor, and positions and orientations of the structures in the OCT measurement data. Thereby, it is possible to display points, which represent the position and orientation of the scanning path in the drawing area 91 such that for each point, the pixel of the image (acquired by the camera sensor), which is shown at the same location in the drawing area 91 as the point, refers to the same portion of the object as the point.

The representation of the scanning path 96 is also transmitted from the controller 29 to each of the displays 41, such that the scanning path is overlaid on the beam paths leading to the eyepieces 13. Thereby, it is possible for the user to view the scanning path 96 in the eyepieces 13 superimposed onto the image of the portion 7 of the object field 11.

In addition to the control elements, which are displayed in the control window 89 of the graphical user interface, the surgical microscope system 1 may comprise further control elements or control units. Examples for such control elements or control units are one or more foot switches, a voice control, or a control by other gestures, such as an analysis of the viewing direction of the user's eyes looking into the eyepieces, for example by using an eyetracker. Thereby, a functionality is provided, which corresponds to a mouse 85, as shown in FIG. 1.

While the foregoing has been described with respect to certain exemplary embodiments, it is evident that many alternatives, modifications and variations will be apparent to those skilled in the art. Accordingly, the exemplary embodiments set forth herein are intended to be illustrative and not limiting in any way. Various changes may be made without departing from the subject-matter defined in the following claims.

1. A Surgical microscope system comprising:
imaging optics for imaging a portion of an object field of the imaging optics onto a camera sensor of the imaging optics, wherein the imaging optics comprises a zoom lens arrangement for varying a magnification of the imaging optics for the imaging of the portion of the object field onto the camera sensor;
an OCT-system for generating an OCT measurement beam for performing measurements by optical coherence tomography;
a beam deflector for deflecting the OCT measurement beam and guiding the OCT measurement beam to selectable locations in the object field;
a graphical user interface for displaying in a drawing area of the graphical user interface an image of the portion of the object field detected by the camera sensor;
a first control module for controlling the beam deflector and the OCT system such that the OCT measurement beam is guided along an adjustable scanning path over the object field and such that OCT measurements are taken at a plurality of locations on the scanning path; and
a second control module for determining the scanning path, wherein the second control module is configured to display the scanning path in the drawing area of the graphical user interface, wherein coordinates of points, which represent the scanning path in the drawing area are determined in dependence on deflecting angles of the beam deflector, which correspond to the scanning path, and further in dependence on the magnification of the imaging optics.

2. The surgical microscope system according to claim 1, wherein the graphical user interface comprises at least one control element for moving the scanning path relative to the object field.

3. The surgical microscope system according to claim 1, wherein the graphical user interface comprises at least one control element for varying a lateral extend of the scanning path relative to the object field.

4. The surgical microscope system according to claim 1, wherein the second control module is configured to adjust a geometry of the scanning path depending on a user input received via the graphical user interface.

5. The surgical microscope system according to claim 1, wherein the graphical user interface is configured to display in the drawing area the plurality of locations on the scanning path, where the OCT measurements are taken.

6. The surgical microscope system according to claim 1, wherein the graphical user interface comprises at least one control element for selecting a scanning path type from a plurality of predefined scanning path types.

7. The surgical microscope system according to claim 1, wherein a scanning path of at least one predefined scanning path types comprises a plurality of scanning lines, which extend linearly and which are laterally spaced from each other.

8. The surgical microscope system according to claim 7, wherein the graphical user interface comprises at least one control element for varying a length of the scanning lines.

9. The surgical microscope system according to claim 7, wherein the graphical user interface comprises at least one control element for varying a lateral distance of the scanning lines.

10. The surgical microscope system according to claim 1, wherein the graphical user interface comprises at least one drawing area for displaying a result of the OCT-measurements.

11. The surgical microscope system according to claim 1, wherein the scanning path comprises a plurality of scanning lines and wherein the graphical user interface comprises a plurality of drawing areas each of which configured to display a result of the OCT-measurements related to one of the plurality of scanning lines.

12. The surgical microscope system according to claim 1, wherein the second control module is configured to determine the coordinates of the points, which represent the scanning path in the drawing area further in dependence on predefined parameters, which influence at least one of a translation of the coordinates in the drawing area and a scaling of the coordinates in the drawing area.

13. The surgical microscope system according to claim 12, wherein the graphical user interface comprises at least one control element for adjusting the predefined parameters.

14. The surgical microscope system according to claim 12, further comprising a calibration object, which comprises a first and a second structure, which are arranged at least one of a predefined position and a predefined orientation relative to each other, wherein the first structure is detectable by the camera sensor and the second structure is detectable by the OCT-system.

15. The surgical microscope system according to claim 14, further comprising a third control module, which is configured to determine at least one of a position and an orientation of the first structure in the image detected by the camera sensor and at least one of a position and an orientation of the second structure in the object field from the OCT measurements and to adjust the predefined parameters in dependence on the determined at least one of the position and the orientation of the first structure, the determined at least one of the position and the orientation of the second structure and the magnification of the imaging optics.

16. The surgical microscope system according to claim 6, wherein the graphical user interface is configured such that the selected scanning path type is adaptable depending on user input via the graphical user interface.

17. The surgical microscope system according to claim 5, wherein the surgical microscope system is configured such that at least one of a number of the OCT measurements and the locations on the scanning path, where the OCT measurements are taken, are settable via the graphical user interface.
